(19) United States
(12) Patent Application Publication
(10) Pub. No.: US 2008/0286751 A1
(43) Pub. Date: Nov. 20, 2008

(54) Dispensing Device For Microfluidic Droplets Especially For Cytometry

(76) Inventors: Philippe Renaud, Preverenges (CH); Nathalie Picollet-D'Hahan, La Ferriere (FR); Vincent Haguet, Grenoble (FR); Francois Chatelain, Beaulieu (FR)

Correspondence Address: THELEN LLP, P. O. BOX 640640, SAN JOSE, CA, 95164-0640, US

(21) Appl. No.: 10/586,831

(22) PCT Filed: Jan. 17, 2005
(86) PCT No.: PCT/FR2005/050025

§ 371 Date: Aug. 7, 2008

(51) Int. Cl. C12Q 1/70; C12M 1/34; C12M 1/36; C12Q 1/02; C12Q 1/68; G01N 15/06; C12Q 1/04; C12Q 1/06; C12Q 1/00

(52) U.S. Cl. 435/5; 435/287.1; 435/288.3; 435/288.7; 435/286.5; 435/29; 435/4; 435/6; 435/34; 435/39; 73/61.71

(57) ABSTRACT

The invention relates to a dispensing device for droplets comprising a first channel (8, 10), known as main channel, for circulation of a first fluid flow, a second channel (12, 13) for circulation of fluid, which forms with the first channel an intersection zone (27) and terminates in an ejection orifice (20), means (4) for measuring a physical property particles or cells in the first channel (18), and means for engendering a pressure wave in the second channel (12, 13).

### TECHNICAL FIELD

The invention relates to a process and a device for manipulating particles in suspension for extracting the particles of interest.

More specifically, the invention relates to a device for analysis and screening of non-marked living cells, and dispensing without contact, and on demand, of droplets of liquid containing the selected cells.

This device enables the depositing of cells onto a substrate, with in particular, a high degree of precision in the positioning of the cells. The invention can be used for example for the production of cell chips comprising one cellular type or different cellular types on the same substrate.

In this respect, the device according to the invention is a very flexible instrument for carrying out automatic detection, counting, analysis, screening and dispensing of particles or cells.

### PRIOR ART

Flow cytometry is a technique used in molecular biology and in cellular biology, and designates analysis of molecular characteristics of cells circulating in continuous flow past a detector. Cytometric analysis allows identification, counting and characterisation of cells or other biological particles (bacteria, parasites, spermatozoids, nodes, chromosomes) as a function of physical-chemical parameters predefined by the operator. The interest of cytometry in flux rests on the simplicity of manipulation of the cells: the capillary through which the cells pass is connected directly to the tank containing the cellular suspension to be characterised.

In addition, since the cells are injected one by one and continuously in front of the detector, the identification speeds of the cells are often considerable and can reach more than a thousand cells per second for certain instruments.

However, the molecular characteristic which is studied must generally be proven by a marking method, and therefore, not really the specified parameter is detected by the apparatus, but the marker associated with it.

The most current marking technique is a fluorescent marker grafted onto a specific molecular component of the cells studied; the fluorescent marker is excited in flow by a laser beam and its response is detected by optical apparatus and characterised by associated electronics.

All the same, previous marking of cells necessitates a preparation stage of the cells prior to their analysis. Fluorescent marking has an interest essentially for cells having very close characteristics and whereof the differences are quasi not detectable by another approach, for example cells of the same cellular type one stem of which is healthy and another is cancerous.

There is however a large number of cases where separation of a heterogeneous cellular suspension can be done on simple criteria of size, membrane and/or cytoplasmic properties, cases for which marking is not necessary.

Non-destructive screening of the cells often follows on from characterisation in flow of cells on the basis of criteria of positivity or negativity defined in advance by the operator. The sampling which is carried out extracts the particles of interest from the solution and collects them in one or more purified fractions in specific receptacles. The technique of cell screening has become a tool in a wide range of fields such as immunology, oncology, haematology, and genetics.

A need therefore exists for an economical system capable of analysing in flow and consecutive screening a wide range of particles, for example non-marked cells coming from solutions having various proteic viscosities and concentrations.

Furthermore, there is a need for tools enabling manipulation of single particles or cells, in particular capable of individually separating each cell from a cellular suspension and positioning each of the cells of interest in a specific site.

A need exists also for a rapid, economical, flexible and easy-to-operate dispensing toll allowing the individual positioning of living cells in localised sites on a two-dimensional network.

All known methods of cellular screening collect all cells responding to the criteria specified in an intermediate receptacle or use steps of cellular concentration by centrifuging, prior to utilising another separation technique for arranging individual cells.

There is therefore a need to eliminate the intermediate stage for collecting purified sub-populations, and for directly separating the cells on the substrate.

All the same, there is currently no system for integrating in one single device the functions of analysis and screening in flow of cells or particles and dispensing of cells or particles of interest on a substrate.

Document EP 1335198 describes a device comprising a channel for flow supplying, a zone for measuring by impedance with a series of electrodes, a zone for screening of particles, and conductive strips for transporting signals to and from the electrodes. The means used for screening the particles is dielectrophoresis with an electrode system.

This known device has a channel with three branches: one fluid entry branch and two outlet branches, the particles being directed to one or the other outlet. The orientation of the particles towards such or such outlet is done necessarily by the electrodes acting by dielectrophoresis on the particles in suspension.

This device is limited to the separation an input fluid in two continuous fluids, whereof one contains screened particles. It does not extract microdroplets from a fluid.

The problem therefore arises of finding a device allowing extraction or ejection of droplets.

An ejection system directed from a carrier fluid is proposed in patent application WO 02/44319, filed by the company Picoliter. However, the means for directing the cells is based on a system of focused waves, typically acoustic waves such as described in application WO 02/054044 filed by the same company. The precision envisageable by means of a device for focusing via acoustic waves is however low, due to the difficulty of focussing an ultrasound wave precisely, reliably and reproducibly.

The problem of finding a device which uses another ejection technique therefore likewise arises.

### DESCRIPTION OF THE INVENTION

The invention aims to resolve these problems.

The invention first relates to a dispensing device for droplets comprising:

a first channel, known as main channel, for circulating of a first fluid flow,

a second channel for circulating fluid, which forms with the first channel an intersection zone and terminates in an ejection orifice,

means for measuring a physical property of particles or cells in the first channel and,

means for creating a pressure wave in the second channel.

The invention therefore relates to a device for dispensing without contact particles or cells, for example on a substrate, the particles being selected by way of triggering means for generating a pressure wave.

The invention therefore permits the selection of particles or non-marked cells in suspension, then the dispense without contact and on demand of particles or cells, for example on a substrate.

The invention differs from components according to the prior art, especially in the means for creating a pressure wave in a supplementary channel, orientation and ejection of particles occurring under the impulse of this pressure wave.

Also, a device according to the invention comprises at least two branches of input channel: one input of the main channel, for a first fluid, and one input branch of the second channel.

The device according to the invention includes, in a reduced space, a system for detection and analysis of particles, for example by impedance measuring, and a microdispenser for the ejection on demand of droplets containing the microparticles of interest.

The device can especially have applications to dilution, or mixing, or concentration, or other applications for screening particles.

According to the invention, the ejection of particles is not bounded to an electric phenomenon applied directly to the particles, but is done by ejection of a micro-droplet under the effect of a pressure wave (regardless of the charge of the particles), with optionally the addition of a second fluid.

The device can further comprise means for analysing the electric signals and for triggering the opening of the means for generating a pressure wave.

In the event where a particle or a cell satisfies specified criteria, triggering of the means for creating a pressure wave can be controlled by a signal or signals originating from these means of analysis or by control receiving signals from the means of detection or measuring.

A microfluidic device according to the invention allows analysis and selection in flow of particles in suspension in a fluid.

The invention allows especially micro-droplets to be extracted from a fluid.

The invention therefore likewise relates to a device allowing the detection, and/or count and/or characterisation in flow of particles or cells, for example non-marked living cells, followed by screening and dispensing of particles or cells of interest on a substrate.

Mixing and dispensing of reagent can be obtained in sites of a substrate prior to, during or after the dispensing of one or more cells on this same site.

This mode of ejection allows the mixing of two liquids (with or without cells present) in well-controlled proportions, in the form of droplets ejected towards a surface.

In the case of dispensing on a surface, one of the two liquids can for example contain reagents which will act on the cell after it is deposited onto a site of the substrate.

Individual cells of one or more cellular types can be placed in a matrix on a substrate to produce cell chips, for example.

Cell chips, that is, two-dimensional networks of living cells, can be produced by means of a device or a process according to the invention by depositing individual cells in wells or holes in a non-planar substrate or in “virtual wells” on a planar substrate. On a cell chip, screening of cells is used on a relatively well-known number of cells on each site and relates to the detection and quantification of a function or a particular characteristic of one cell among a population of cells of different cellular types and/or at different stages of the cycle of cellular division. The cellular function or characteristic studied is highlighted by the effect of chemical, optic, electric stimulus, . . . present in the site or generated outside towards the chip. On a non-planar substrate the density of sites on the chip depends on physical specifications such as the thickness of the walls between two wells or two holes, and/or the space left between two sites for possible microfluidic connections.

A much higher site density can be reached by introducing the cells to virtual wells made in the form of drops deposited on a planar substrate.

In the case where the dispensing sites correspond to wells, for example virtual wells on a substrate, the invention is capable of providing very high-density cell chips, that is, at densities much greater than accessible from the positioning of cells in well plates or in holes in the substrate.

A system according to the invention can further provide a device such as hereinabove and a carrier-substrate plate allowing a substrate to be shifted in X, in Y and in Z with a high degree of precision.

Accordingly, the ejected particles can be placed on the substrate.

Optionally, an enclosure controlling the atmosphere encloses the whole device or system.

The device according to the invention can also be used as an instrument for the production of chips and for any other application requiring a particular spatial arrangement of a controlled number of cells on a substrate.

For example, the ability to precisely deposit a varied number of products and objects such as biopolymers, cells of different types and growth factors (stimulants and inhibitors) can prove to be advantageous within the framework of regeneration of damaged tissue and the creation of artificial tissue.

The device according to the invention also helps to detect and analyse in flow non-marked cells, and integrates the additional function of dispensing a controlled and reproducible number of cells on sites.

The sedimentation of cells in the device is avoided due to continuous circulation of the cells in the device according to the invention, whereas the input of cellular aggregates in the device according to the invention can be prevented by the small dimensions of the device.

The dispensed volumes can be reduced to the minimal volume necessary to contain a microparticle, allowing the deposit of each cell or particle to be localised with precision and cell chips having a very high density of sites to be produced.

For example, droplets with volume between around 1 femtolitre and 10 μL, or of a diameter of around 0.1 μm to 2 mm or 5 mm can be generated.

Evaporation of the medium containing the cells can be limited by using atmosphere control, in particular control of the degree of hygrometry.

According to a particular embodiment, a device according to the invention comprises two branches of main channel, and at least one branch of secondary channel, joining up in the intersection zone.

Furthermore, an input opening of the first fluid flow can be connected to a first branch of the first channel, and an injection opening of another fluid flow can be connected to the second channel.

The device according to the invention can comprise at least one layer deposited on the surface of a first and/or second substrate or at least an intercalary film inserted at the interface of a plate and a platform, channel branches being hollow or pierced in said layer(s) or in said intercalary film(s).

At least one opening and/or orifice can be pierced through the thickness of the substrate of the platform and/or the plate.

Each layer deposited on the substrate surface or each intercalary film inserted at the interface of the platform and the plate can be composed of one or more materials chosen from the group of materials comprising: etching materials of the electronics field, resins, polymers, dielectric materials, insulating compounds of semiconducting elements, especially photosensitive or electrosensitive resins, polyimide, polystyrene, polyethylene, polyurethane, polyvinyl, poly-dimethylsiloxane, nitrides, oxides and silicon compounds, as well as glass.

Channel branches can form capillaries of transverse dimensions of the order of several tens of nanometres (for example 20 nm) to a few millimetres (for example 2 mm or 5 mm).

Measuring means can be of the optical and/or electric type, for example means for measuring the impedance of the fluid medium. This can be realised with a series of electrodes, for example arranged along at least one channel branch.

For example, at least three microelectrodes are arranged in a channel branch to measure a differential variation of impedance.

Means for creating a pressure wave can comprise an electrovalve and/or a physical-mechanical actuator for generating a pressure wave.

A device according to the invention can be made by assembling a microfabricated chip comprising microchannels and a series of microelectrodes, and an electrovalve which generates a pressure wave causing the ejection of particles outside the chip.

The ejection volume at the outlet of an electrovalve can be very precise and reproducible.

The invention likewise concerns applications relating to the production of micro-droplets, whereof the composition in particles was able to be adjusted; this technique allows dispensing without contact with individual control of each particle, and with micro-droplets volumes of one to several orders of magnitude less than those of the prior art.

According to an exemplary embodiment, a first fluid circulating in the device comprises for example a liquid, or a solution, or a suspension or a medium containing particles or biological cells, or components or cellular products, especially bacteria, or cellular lines, or globules, or cellular nodes, or chromosomes, or DNA or RNA strands, or nucleotides, or ribosomes, or enzymes, or protides, or proteins, or parasites, or viruses, or polymers, or biological factors, or stimulants, and/or growth inhibitors.

Particular examples of the particles are solid particles insoluble in the liquid, such as: dielectric particles (latex microballs for example), or magnetic particles, or pigments (ink pigments for example), or colorants, or protein crystals, or powders, or small polymeric structures, or insoluble pharmaceutical substances, or small size aggregates (“clusters”) formed by colloid agglomeration.

The second flow comprises for example means for reaction or interaction with the first fluid, especially at least a reagent, an active ingredient, a marker, a nutrient medium, a chemical product, an antibody, a DNA sequence, an enzyme, a protide, a protein, a biological factor, a stimulant or a growth inhibitor.

### BRIEF DESCRIPTION OF THE DRAWINGS

Other objectives, characteristics and advantages of the invention will emerge from the following description of embodiments, given by way of non-limiting examples, in relation to the attached diagrams, in which:

FIGS. 1A and 1B illustrate open views, when the wafers are disassembled, of a dispensing device for droplets according to the invention;

FIG. 2 illustrates a top plan view, after assembly of the wafers, of a device according to the invention;

FIG. 3 illustrates a frontal view of the whole of the device mounted with an electrovalve and a counterweight, according to the invention;

FIG. 4 illustrates a bottom plan view of the device according to the invention;

FIG. 5 illustrates an overall view of a system connecting a device and a tracing table in an enclosure, according to the invention;

FIGS. 6A to 6D illustrate alternative embodiments of a device according to the invention.

### DETAILED DESCRIPTION OF EMBODIMENTS OF THE INVENTION

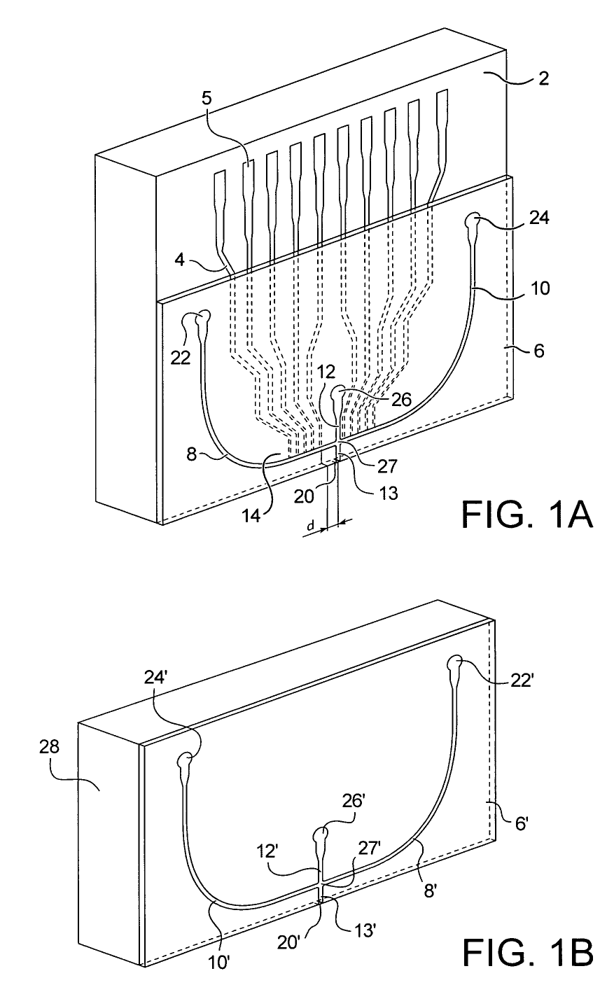

A first example of a device according to the invention will now be described in conjunction with FIG. 1.

In this Figure, the device comprises a substrate 2, for example made of glass, on which a series of electrodes 4 is formed.

A layer 6 at least partially covers the electrodes. This layer is for example made of polyimide or any other material capable of being deposited in the form of a thin layer, in particular any photosensitive or electrosensitive resin such as, for example, S1818 or S1813 resins as marketed by Shipley or electrosensitive polymethylmethacrylate resins. Formed in this layer are a first and a second part 8, 10 of a first microchannel and a first and a second part 12, 13 of a second microchannel, as well as an opening 20, or ejection orifice.

A portion 14 of the microelectrodes is in contact with the first channel 8, 10.

Defined at each end of the branches of the first channel 8, 10 are wider zones 22, 24, which will serve as input and outlet points of a fluid circulating in the channel 8, 10.

Similarly, an opening 26 will act as input point to a second fluid for circulating in the second channel 12, 13, toward the ejection orifice 20.

An intersection zone 27 is located in between the input point 26 of the second channel 12 and this ejection orifice 20.

In FIG. 1A the parts 12, 13 of the second microchannel are of a dimension or cross-section comparable to the main microchannel 8, 10, and cross the latter substantially at a right angle. But intersection at an oblique angle can also be achieved. The second microchannel 12, 13 connects the ejection actuation device to the orifice 20 for ejection of the droplets.

The expression “propulsion channel” consequently designates the part 12 of the second channel which extends from the introduction zone 26 to the main channel, and the expression “ejection channel” designates the channel part 13 which extends from the main channel 8, 10 to the ejection orifice 20.

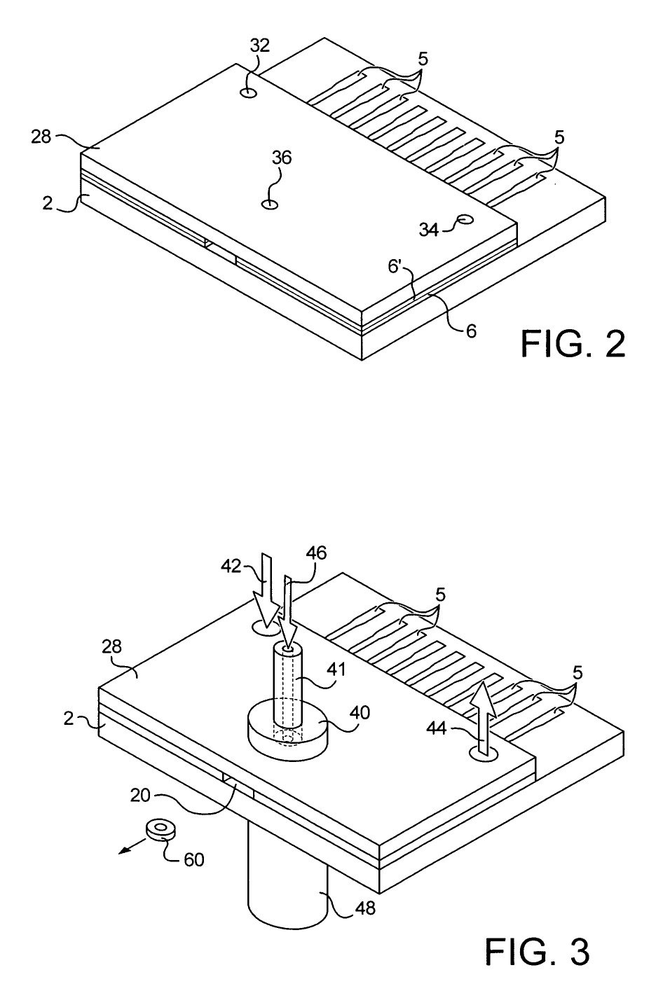

The layer 6 is aimed to be covered by a second substrate 28, for example also made of glass, as illustrated in FIG. 2.

FIG. 1B illustrates this second substrate 28. This second substrate 28 is preferably provided with a layer 6′ similar to the layer 6 of the first substrate 2, fitted with patterns 8′, 10′, 12′, 13′, 20′ reproducing the channels 8, 10, 12, 13, 20 and with zones 22′, 24′, 26′ reproducing the fluid input and outlet zones 22, 24, 26.

The device is assembled by turning the wafer of FIG. 1B over onto that of FIG. 1A.

In combination with the first layer 6 the second layer 6′ enables an efficient assembly of the substrates 2, 28.

The second substrate 28 is provided with three orifices 32, 34, 36, which communicate with the openings 22, 24, 26 defined in the layers 6 and 6′ (FIG. 2).

FIG. 3 illustrates a view in perspective of the device, arrows 42, 44 symbolising the input and the outlet of a first fluid, for example a cellular medium. This Figure likewise illustrates means 40, 41 (here: an electrovalve) for applying a pressure wave in the channel 12. These means are illustrated as placed against the substrate 28. The arrow 46 symbolises the introduction of a second fluid via these means 40, 41 to the channel 12. A counterweight 48 can optionally be fixed against the opposite substrate 2.

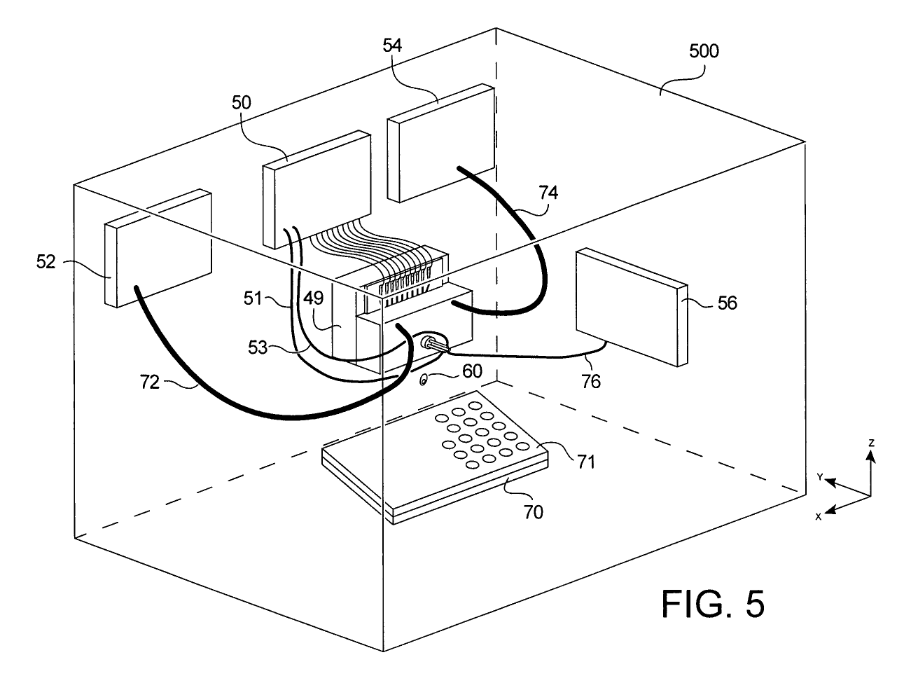

The electrodes 4 can be attached by electrical connections 5 to analysis means 50 (FIG. 5), for example an electronic circuit. These means are configured or programmed to detect the passage of some particles or cells at the level of the electrodes 4, in the portion 14 of the latter which traverses the first channel 8, 10 (see FIG. 1).

Advantageously, the electrodes 4 and the electronic means 50 constitute an analysis device by impedance measuring.

Moreover, if the substrates 2, 28 are transparent, optical detection means can be used, directed towards the chip. A signal produced by these optical means can be sent to the control means 50 and used for triggering the ejection means 40, for example alone or in combination with the signals emanating from the electrodes 4. Therefore, optical detection techniques can be sued by way of optical means directed between the electrodes 14 of the device. These optical means function for example on the principle of optical diffusion as the cells or particles pass.

It is likewise possible to conduct optical analysis alone without employing the electrodes. In this case, a device according to the invention does not necessarily comprise electrodes.

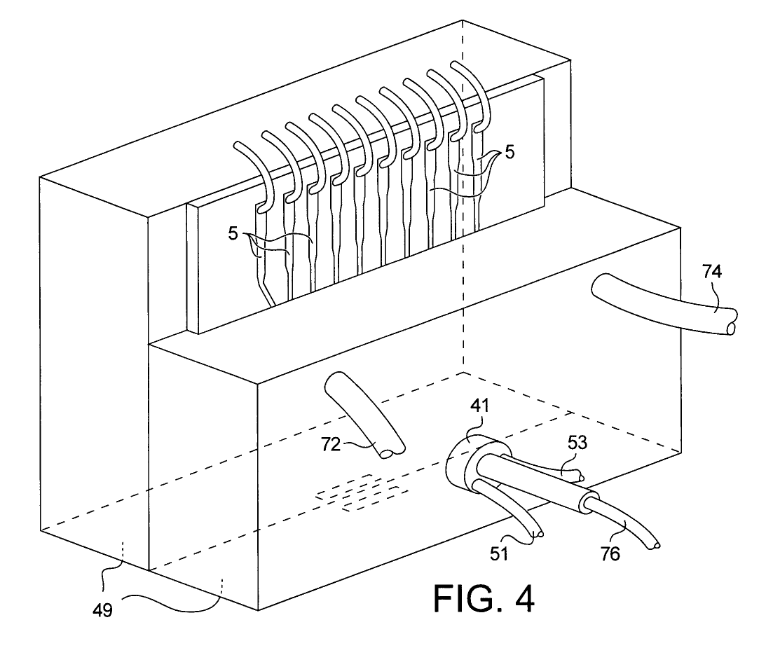

FIG. 4 illustrates a view of the device in a box or mould 49, for example made of plastic. Reference numerals 72, 74, 76 designate fluid connections, and reference numerals 51, 53 designate electrical connections.

The system can be continuously supplied with fluid from a tank 52 (FIG. 5) containing, for example, a homogeneous or heterogeneous cellular suspension. The fluid or the liquid passes through the device via the channel branches 8, 10, exiting by the second opening 24 and is collected in a second tank 54. A tank 56 contains the fluid which circulates in the means 40, 41 and towards the channel 12.

The main microchannel 8, 10 preferably has a cross-section adapted to the type of particles which must be ejected, for example between one micrometer and three hundred micrometers for cells, and in some way forms a capillary. Therefore the cells, circulating in the main microchannel 8 pass in front of the electrodes 14 and are analysed one by one, for example by impedance measuring, their electric properties being measured in flow by means of electrodes 14. A series of three very close microelectrodes allows a differential variation of impedance to be measured during passage of a particle and thus allows the particle to be identified by comparing the measurement to the expected impedance profile, as described in the patent application EP 1335198 and in the document by Gawad S, Schild L and Renaud Ph, <<Lab on a chip>> 2001, 1: 76-82.

It is thus possible to identify cells or particles according to pertinent characteristics, detected electrically and/or optically, in particular by criteria of size, cytoplasmic conductivity and/or membrane capacitance.

This technique is very sensitive and detects, for example, the influence of cytoplasm or significant differences for microballs whereof the diameter differs by just a few microns. Operations for processing registered electric signals can be conducted simultaneously by the electronic circuit 50 to be able to detect and characterise the particles in real time, as well as numbering each identified category of particles.

This method further allows the measure of the speed of particles as they pass in front of the electrodes 14. Depending on the measure results, the device can be programmed for parametering an ejection decision case by case, as explained herein below.

In fact, means 50 can be configured or programmed to send, depending on the achieved measurements, a command or a signal to the means 40. The latter will then generate a pressure wave which, as it is transmitted to the fluid contained in the channel 12, will push towards the ejection orifice 20 the fluid coming from channel 8 and located in the intersection zone 27 The channel 12, 13 effectively connects the ejection actuation device 40 to the ejection orifice 20 of the droplets.

The means 40 are situated set back relative to the main channel 8, 10 so that they do not risk being clogged or damaged by the first fluid circulating in the main channel 8, 10, or by the accumulation of cells and proteins of a cellular medium contained in the first fluid. In fact, the second fluid in the propulsion channel 12 then acts as an interface with the fluid or the cellular medium circulating in the main channel 8, 10.

The fact that the means 40 are set back vis-à-vis the main channel 8, 10 further helps reduce the shear stress on the cells during ejection and centres the major part of the generated wave towards the ejection channel 13.

On the other hand, if the dimensions of the main microchannel 8, 10 are reduced, aggregates of cells, which could clog the microdispenser, cannot accede to the means 40.

A high degree of precision in the ejection decision can be reached due to the slight distance d between the series of electrodes 14 and the intersection zone 27 (FIG. 1), for example between 5 μm and 15 μm, for example equal to around 10 μm. The series of electrodes is therefore placed as close as possible to the intersection zone 27.

As illustrated in FIG. 3, the dispensing of particles is therefore achieved by the ejection from the device of a droplet 60 comprising the particle of interest, for example to a site of a substrate. The relative position of a device according to the invention and of such a substrate 71 is indeed illustrated in FIG. 5. Ejection of the particles of interest allows screening of the particles depending on criteria predetermined by the operator.

Detection and ejection of the particles of interest can be coordinated by way of means 50 which can analyse in real time the electric signals measured between the electrodes 4. In particular, the width and/or the instant of triggering and/or the form and/or the intensity of a control signal can be adapted by the means 50. As a consequence, each droplet 60 produced contains a microparticle of interest, and can be ejected to a particular receptacle or to a particular site on a substrate.

A process for on-demand dispensing according to the invention therefore uses a pressure wave operated, in the given example, by a miniature valve 40 (FIG. 3) electrically controlled by a microsolenoid, the whole being controlled by the means 50.

According to a particular embodiment an electrovalve can be integrated directly onto the chip by microfabrication techniques. Other means can also be used for generating the pressure wave at the site of the electrovalve. For example, on-demand dispensers of piezoelectric type, or acoustic type, or electromechanical type, or pneumatic type, or actuated by an air or solvent bubble can be used, replacing the miniature valve at the end of the propulsion channel 12 by a piezoelectric material, or an electroacoustic transducer, or a mechanical actuator, or a piston, or heating resistor.

In the case of on-demand dispensing of thermal type, the relative removal of the heating resistor (due to the channel portion 12 which separates the means 40 from the main channel 8, 10) avoids damaging the cells and thus favours their survival rate.

When the means 50 detects a particle which verifies specified criteria, a pressure pulse is applied by the means 40 and a droplet 60 is ejected via the ejection orifice. The pressure wave causing the ejection is generated by a second fluid or liquid 46 propelled by the means 40 (FIG. 3). This fluid, or liquid, is conveyed through the propulsion channel 12 vis-a-vis the ejection channel 13, passes through the main channel 8, 10 and is expelled through the ejection orifice 20. Via this movement, a portion of liquid is extracted from the main channel and pushed towards the ejection orifice 20 in a direction substantially perpendicular to its initial displacement, or in another direction if the ejection channel and the main channel do not cross at a right angle. The volume element ejected from the main channel contains the fraction of interest only, in particular the volume element which comprises the cell or particle of interest.

Beyond any ejection, the ejection channel 13 is filled by capillary action. The liquid is retained by its surface tension at the ejection orifice 20. This second liquid, propelled by the means 40, is initially at rest in the propulsion channel.

Applying a pressure pulse produces the ejection of a droplet whereof the volume is fixed by the shape and duration of the pulse.

For an identical pulse, the same volume is dispensed irrespective of the density, the viscosity and the surface tension of the liquid and the possible variations of atmospheric conditions.

The dispensed volume can be precisely monitored by adjusting the triggering instant and/or the opening duration and/or the form and/or the intensity of the pulse electric controlling the means 40 thanks to the means 50, for example programmed to this effect. The ejected volume is for example between 0.1 pL and 10 μL, depending on the dimensions of the channels and the pulse parameters of the electrovalve.

A mode for using the microdispenser according to the invention is therefore the production of droplets each of which contains a microparticle of interest.

The microdispenser according to the invention integrates the functions of loading the liquid, analysing microparticles or non-marked living cells, separating and ejecting the cells or particles of interest, and discharging the liquid with the refused cells or particles.

The microparticles of interest can be detected and analysed in flow by impedance measuring (electric detection) and/or by optical detection upstream of the ejection zone. The mode of ejection of the droplets can be of the <<Drop On Demand>> type (DOD) and without contact, producing a dispense from a flow, of the volume element of interest, that is, containing a selected microparticle.

FIG. 5 illustrates a carrier-substrate system 70, of tracing table type which registers the shifts of a substrate 71 in X, in Y and in Z, with a given precision (preferably micrometric), in view of receiving the droplets 60 which contain each a cell on adequate sites of a substrate. The shifts of the tracing table can be coordinated with ejections of droplets, by way of means 50. In particular the positions of the carrier-substrate plate under the ejection nozzle can be controlled depending on the type of cell or particle detected. Identification of the microparticle by way of means 50 at the same time enables the dispensing or ejection operation and the positioning of the site on the substrate.

Another mode of utilisation of a device according to the invention is the ejection of a series of droplets, one of which contains the microparticle. The alignment of the dispensing head and the substrate 71 helps to control the number of droplets deposited in each site and, if necessary, to later add new droplets on the dispensed sites.

The concentration of particles on a site of the substrate depends on the number of droplets dispensed, in particular the concentration of particles can be less or more than the initial concentration in the tank.

Also, the device allows adding locally or regularly solvent and/or reagents with a high degree of precision for example within the scope of experiments dependent on time.

The liquid 46 propelled by the means 40 can be the same or not as the liquid in the main channel 8, 10 which contains the particles. A possibility for mixing two liquids at the time of ejection is included when the two liquids are different or if the liquid propelled by the electrovalve contains a specific product.

This mixing function enables for example reagents to be introduced to the droplet containing a cell, which will act on the cell after deposit on the site. Later mixing is likewise possible with the same device by depositing fresh droplets on already existing sites on a substrate 71.

By way of example, the reagent can be active ingredients, immunofluorescent markers targeting specific antigens, markers of metabolism or viability such as trypan blue, or toxic products, or DNA sequences for transfection of cells.

The reagent can likewise be proteins, for example enzymes such as trypsin. The deposit of two cellular types on the same site allows cell-cell interaction study. The dispenser can likewise be connected upstream or downstream to other devices for analysis in flow, such as for example capillary electrophoresis and/or mass spectroscopy.

The microdispenser according to the invention can be made by means of conventional techniques of microfabrication in a clean room. An example of the production process will now be described.

A first optical mask is used to produce the patterns of the microelectrodes in a photoresin (available for example under the commercial reference “AZ5214”) on a wafer made of glass, with for example four inches (or about 10 cm), and deposit a metallic bilayer of 50 nm titanium and 150 nm platinum by cathodic pulverisation.

The microelectrodes 14 have a width of 20 μm, an inter-electrode distance of between 20 μm and 50 μm, and extend as far as electric contact blocks 5 situated on the opposite side of the chip (FIG. 1).

The microdispenser can be made by assembling two chips identical to one another, with the difference that microelectrodes 14 can be made only on one of them. The microelectrodes can therefore be produced on one side only of the glass wafer, for example the left side, while no electrode is made on the right side.

The microchannels are defined in a polyimide photoresin (PI-2732, Dupont) owing to a second optical mask. The dimensions of the main microchannel 8, 10, of the propulsion channel 12 and of the ejection channel 13 are preferably similar in the intersection zone 27, with a cross-section adapted to the type of particles which must be ejected. The width of the microchannels is typically between 1 μm and 300 μm. The height of the channels, determined by the thickness of the layers 6, 6′ of polyimide, can be between 100 nm and 75 μm; since the microdispenser can be obtained by assembling two chips of identical thickness, it is enough that the thickness of polyimide deposit be equal to half of what is wanted (between 50 nm and 38 μm).

The thicknesses of polyimide 6, 6′ can be between 15 μm and 25 μm, and the thicknesses of the channels obtained in this layer 6, 6′ can be between 30 μm and 50 μm, whereas the widths of the channels are between 50 μm and 100 μm in the crossing zone 27 of the microchannels. The glass wafer is cut out in two pieces, one carrying the microelectrodes, the other carrying none. The two pieces are aligned on one another to form the microchannels and assembled by thermal annealing at 300° C. under nitrogen atmosphere.

The chips are cut out to separate the microdispensers and disengage the blocks 5 of the microelectrodes. Three openings are produced in each device by electroerosion with a tungsten tip: two openings at each end of the main microchannel form the input and the output of the solvent containing the microparticles, the third opening near the centre of the microdispenser is dedicated to positioning the electrovalve. In one configuration, the opening 36 for the electrovalve is made on the glass wafer 2 which carries the electrodes 14, 4, 5. In another configuration illustrated in FIG. 2, the opening 36 for the electrovalve 40 is made on the glass wafer 28 opposite the wafer 2 which carries the electrodes 14, 4, 5 and which forms the platform. Each of the openings 32, 34, 36 can be made to any side of the microdispenser, in particular the openings 32, 34 for the input and the output of the solvent and the opening 36 of the electrovalve can be situated to the same side or placed opposite.

The used electrovalve is for example a microdispensing valve VHS Small Port INKA 4026212H (The Lee Company, Westbrook, USA), with an outlet orifice of 100 μm in internal diameter, kept against the chip by a tight gasket of polydimethylsiloxane (PDMS) or with a O ring made of plastic. The electrovalve-chip assembly is stabilised by keeping a counterweight 48 on the chip on the opposite side of the electrovalve, which firmly supports the chip in the vertical plane and ejects droplets downwards. The electrovalve 40 and the counterweight 48 are supported in the direction orthogonal to the plane of the chip due to a plastic mould 49 which encases the chip, the electrovalve, the counterweight and the fluid connections to the tanks.

The design of the microdispenser is preferably symmetrical relative to the axis formed by the propulsion 12 and ejection channels 13: the channels 8, 10 and the microelectrodes 14 are reproduced identically relative to this axis of symmetry. In this configuration, the input and the outlet are interchangeable since detection of the microparticles can be made from both sides of the microdispenser. Also due to the presence of microelectrodes 14 after the ejection channel 13, the shifts of the cells or particles which have not been selected for ejection can be followed.

Optical tracking of the shifts of the microparticles can likewise be done via both faces of the microdispenser, when the latter is made from a glass wafer (transparent material). In particular, optical observation of the microparticles is useful during the first adjustments made to coordinate electrical detection of the cells or particles and triggering of the opening of the electrovalve.

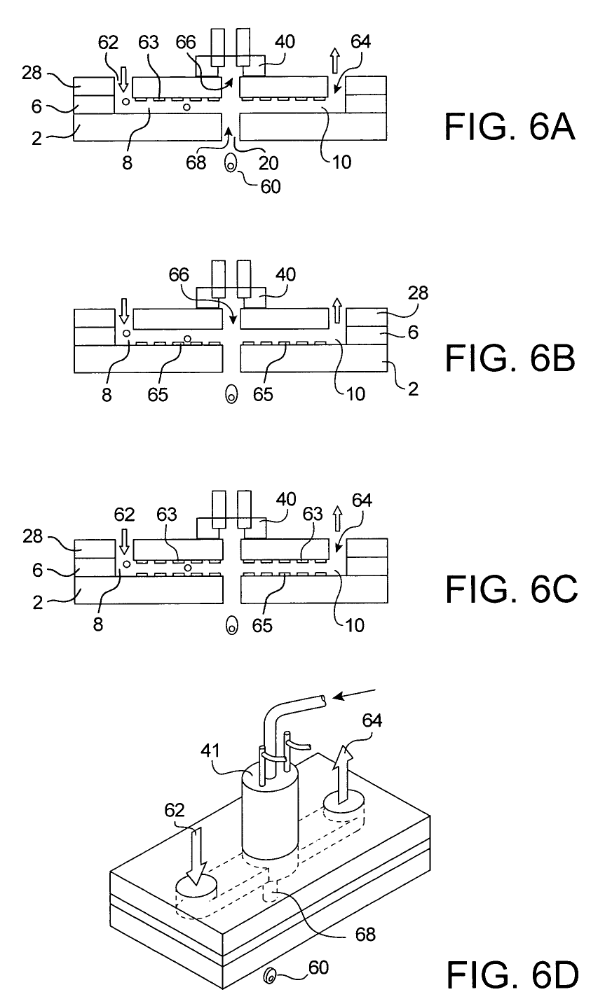

In another configuration illustrated for example in FIG. 6C, the microelectrodes 63, 65 are arranged on both sides of the microdispenser. In this case, the difference in impedance is measured between two opposite electrodes 63, 65 in vis-à-vis, as specified in the document by Gawad S and coll., “Lab on a chip” 2001, 1: 76-82.

In another configuration, the electrovalve 40 is placed above the main microchannel, and ejection 66 and propulsion channels 68 consist of openings made through the glass substrates 28 and 2, as illustrated in FIGS. 6A to 6D. In this case, the ejection of droplets 60 is produced directly in the axis of the electrovalve 40.

Several types of materials are possible for the substrates of the microdispenser and for making the microchannels, but the used materials are preferably insulating (so as not to perturb electrical analysis) and biocompatible: photosensitive or electrosensitive resins, polymers (polystyrene, polyethylene, polyurethane, poly(dimethylsiloxane) (PDMS), poly(vinyl chloride), . . . ), insulating layers deposited by Chemical Vapor Deposition (CVD) such as layers of Si₃N₄ or SiO₂, . . . .

One possibility is to make the microchannels directly in the glass substrate by chemical etching with diluted BHF (buffered hydrofluoric acid) or HF (hydrofluoric acid) and to seal the two chips by adhesion or by direct welding.

The invention described is very flexible and adapts to the specifications wanted by the user. In particular, the device can be utilised for one part only of its functions.

For example, one application is simple counting in flow of cells without later ejection. Another possible operation is characterisation and counting in flow of cells without ejection. Counting can be done by way of means 50 which counts the particles or the cells having given characteristics, measured by electric and/or optical measuring means such as described hereinabove.

An application is dilution of particles: the droplets 60 are formed by mixing the liquid circulating in the first channel and the liquid propelled by the system producing the pressure wave (for example, electrovalve). The volume of the droplets is proportional to the time for opening the system producing the pressure wave. Dilution can thus be undertaken by increasing the volume of the ejected droplet.

Another possibility for dilution relates to the droplet deposited on the substrate, constituted by several smaller droplets ejected by the dispenser. Some of the ejected droplets can correspond to “empty” droplets, that is, droplets without cells or particles, which increase the volume of the droplet on the substrate and therefore dilute the components which are contained therein.

Another application relates to screening and ejection of cells to one or more receptacles without specific arranging of the cells on a substrate.

More generally, screening relates to the possibility of letting cells or particles circulate to the outlet 24 of the first channel (FIG. 1A) or to propel them outside the dispenser in the form of droplets. Separation of the cells or particles to two outlets is therefore completed at the intersection 27 of the two channels, depending on the signals registered by the detection system.

Another form of screening consists of positioning a substrate 71 depending on the cell contained in the ejected droplet so that the cells are collected in different receptacles according to cellular type. This produces purified cellular cultures in specific receptacles.

The invention supplies precise numbers of cells to receptacles or to sites determined on a substrate, for example for applications for screening and/or distribution and/or dosage and/or transfer of samples.

The microdispenser can be used for extracting one or more cells from the medium with the possibility of precisely monitoring the volume of containing liquid, and consequently concentrating the cells on the site or diluting them by addition on the site of additional supplementary droplets without cells.

Blanking the cells on a substrate can relate to one or more cells on each site. In the case of sites containing several cells, a single cellular type or several can be deposited on the same site.

Droplets without cells can be added at will to sites of the substrate, whether they contain cells or not, for example for delivering reagents in a very precise quantity to the site or for compensating the evaporation of the liquid on the substrate.

Advantageously, the reduced size of the device (1 cm to 3 cm side) allows manipulation of reduced volumes of cellular suspension, for example for taking rare or precious samples, or for extracting a very small quantity of cells diluted in a relatively big volume of liquid. The initial cellular concentration in the tank can be low and adapted to the total number of cells which has to be dispensed, enabling dispensing cells from small samples extracted from more significant cellular media. The microdispenser can possibly dispense all the cells of interest of a medium, if necessary by utilising a loop connection of the outlet and the input of the dispenser so as to make several successive passes of the medium in the device (for example if the number of cells is high and does not trigger all the ejection operations during a single pass).

As shown in FIG. 5, the whole of the apparatus 49, 50, 52, 54, 56 of the device and of the tracing table 70, 71 is placed in an enclosure 500 for atmospheric control, that is, control of humidity, pressure and temperature. Greater reliability and higher precision are reached during production of the spots due to reduction in movements of air during dispensing. In addition, monitoring environmental conditions generates a degree of air hygrometry greater than 80% and thus minimises evaporation of the drops on the sites of the substrate. In general, evaporation of the drops of cellular medium in ambient air is around 3 nL/min for dispensed volumes of the order of 10 nL, and therefore the use of air highly loaded in humidity helps store the drops produced for at least two weeks without major variation in their volume.

1. Device for dispensing droplets comprising:
a first channel, known as main channel, comprising at least one branch, for circulation of a first fluid flow,
a second channel for circulation of fluid, comprising at least one branch, which forms an intersection zone with the first channel and terminates via an ejection orifice,
measuring means of a physical property of particles or cells in the first channel, and
means for generating a pressure wave in the second channel.

2. Device according to claim 1 comprising two branches of the main channel, and at least one branch of a secondary channel, which join up in the intersection zone.

3. Device according to claim 2, comprising an input opening of the first fluid flow connected to a first branch of the first channel, and an injection opening of another fluid flow in the second channel.

4. Device according to claim 1 wherein the second channel comprises branches on either side of the main channel.

5. Device according to claim 1 wherein, apart from the ejection orifice of droplets which provides an outlet for part of the flow, the device comprises another outlet orifice for another part of flow.

6. Device according to claim 1 comprising a platform of a first substrate and a plate of a second substrate covering the platform, in which, or on which, or between which, or at the interface of which, the channel branches are arranged.

7. Device according to claim 6 further comprising at least one layer deposited on the surface of the substrate(s) or at least an intercalary film inserted at the interface of the plate and of the platform, the channel branches being digged or pierced in said layer(s) or in said intercalary film(s).

8. Device according to claim 7 wherein each layer deposited on the substrate surface or each intercalary film inserted at the interface of the platform and of the plate are composed of one or more materials selected from the group of materials comprising etching materials of electronic field, resins, polymers, dielectric materials, insulating compounds of semi-conductor elements, especially photosensitive or electrosensitive resins, polyimide, polystyrene, polyethylene, polyurethane, polyvinyl, poly-dimethylsiloxane, nitrides, oxides and silicon compounds, as well as glass.

9. Device according to claim 7 wherein at least one opening and/or one orifice are pierced through the thickness of the substrate of the platform and/or of the plate.

10. Device according to claim 6 wherein the substrate of the platform and/or of the plate is made of glass.

11. Device according to claim 10 wherein at least one orifice and/or one opening and/or at least one channel branch are pierced or digged in the substrate.

12. Device according to claim 1 wherein the channel branches form capillaries having transversal dimensions of the order of a few tens of nanometres to a few millimetres.

13. Device according to claim 1, comprising means for measuring optical properties of a fluid flow and/or means for measuring the impedance of the medium and/or means for measuring electric parameters of a fluid flow, which preferably are placed near the intersection zone.

14. Device according to claim 1, the measuring means comprising a series of electrodes, which preferably are arranged along at least one channel branch.

15. Device according to claim 14 wherein matching electrodes are arranged on either side of a channel branch.

16. Device according to claim 1 wherein at least three microelectrodes are arranged in a channel branch for measuring differential variation of impedance.

17. Device according to claim 1 further comprising an electrovalve and/or a physical-mechanical actuator for generating a pressure wave and/or a flow.

18. Device according to claim 1, comprising control means for receiving measuring signals originating from means for measuring physical properties, and for sending a control signal to the means for generating a pressure wave.

19. Device according to claim 18 wherein the control means are suitable for controlling the amplitude and/or the triggering instant, and/or the form, and/or the duration of the control signal.

20. Device according to claim 1, further comprising means for supply and/or continuous circulation of fluid in the main channel and/or at least one tank connected to a respective channel branch for containing a liquid, in particular a liquid or a medium comprising a solution or a cellular suspension.

21. Device according to claim 1, further comprising a confinement enclosure.

22. System for depositing at least one sample on a substrate comprising a dispensing device for droplets according to claim 1 associated with means for scanning or relatively shifting of the substrate and the dispensing device so that the ejected droplets can be deposited from place to place on the substrate.

23. Method for dispensing droplets, making use of a first channel and a second channel which forms an intersection zone with the first channel and terminates in an ejection orifice, the process comprising the steps consisting of:
circulating a first fluid in the first channel;
measuring a physical property or analysing the contents of the flow of the first fluid; and,
generating a pressure wave as depending on the results of the preceding step.

24. Method according to claim 23 comprising a step consisting of injecting a second fluid flow in or towards the intersection zone together with the steps of generating a pressure wave, the second flow being different from or identical to the first fluid in composition.

25. Method according to claim 24, comprising steps consisting of:
supplying an input opening of a branch of the channel with a continuous flow of the first fluid;
feeding an injection opening communicating with the intersection zone by a flow of the second fluid.

26. Method according to claim 25 wherein dilution or a mixture of the first fluid and of the second fluid is collected at an outlet opening of the channel or at the droplets ejection orifice.

27. Method according to claim 24 wherein the second flow comprises means of reaction or interaction with the first fluid, especially at least a reagent and/or an active ingredient, and/or a marker, and/or a nutrient medium, and/or a chemical product, and/or an antibody, and/or a DNA sequence, and/or an enzyme, and/or a protide, and/or a protein, and/or a biological factor, and/or a stimulant, and/or a growth inhibitor.

28. Method according to claim 23, comprising a step consisting of:
delivering an electrical command calibrated for injecting the second fluid flow to the intersection zone, the electrical command having an amplitude and/or a triggering instant and/or a form and/or a pulse duration controlled to eject a droplet of controlled or calibrated volume.

29. Method according to claim 23 wherein a concentration, separation, and/or extraction, and/or selection and/or collection of components of the first fluid is gathered at the ejection orifice of droplets or at the outlet opening of the channel.

30. Method according to claim 23 wherein the first fluid comprises a liquid, or a solution, or a suspension or a medium containing biological cells, and/or components and/or cellular products, especially bacteria, and/or cellular lines, and/or globules, and/or cellular nodes, and/or chromosomes, and/or strands of DNA or RNA, and/or nucleotides, and/or ribosomes, and/or enzymes, and/or protides, and/or proteins, and/or parasites, and/or viruses, and/or polymers, and/or biological factors, and/or stimulants, and/or growth inhibitors.

31. Method according to claim 23 wherein the first fluid comprises a liquid or a solution, or a suspension, or a medium containing particles which are preferably solid particles insoluble in liquid, such as dielectric particles or electric particles, or magnetic particles, or pigments, or dyes, or protein crystals, or powders, or polymer structures, or insoluble pharmaceutical substances, or clusters or aggregates of small size formed by agglomeration of colloids.

32. Method according to claim 23, comprising an intercalary step consisting of:
triggering an ejection control pulse of droplets as a content of interest passes.

33. Method according to claim 23, comprising an intercalary step consisting of:
undertaking cytometric analysis of the flow of the first fluid to detect biological particles or cells, cellular components or products contained in the flow; and,
triggering an ejection control pulse of droplets for isolating the biological particles or cells, the cellular components or products which have been detected.

34. Method according to claim 23, the measuring step comprising at least measuring one electric parameter and/or measuring impedance or differential variation of impedance of the contents of the flow of the first fluid circulating in the channel, and/or optical measuring of the contents of the flow of the first fluid circulating in the channel.

35. Method according to claim 23, wherein droplets of low volume are generated, especially of the order of a femtolitre to a microlitre, or of a micrometric diameter, of the order of 0.1 μm to a few millimetres.

36. Method according to claim 23, wherein droplets of fluid are ejected from place to place on a substrate or a support.

37. Method according to claim 23, being used in a confined atmosphere.

38. Method according to claim 23, applied to extraction, selection and/or screening of cellular lines; and/or to detection, and/or identification, and/or counting and/or characterisation of biological particles or cells, components or cellular products, and/or to the deposition of particles or biological cells, or cellular components or products, on preferred sites of implantation and/or growth and/or regeneration and/or grafting, especially cellular lines, or biopolymers, or biological factors, or stimulants or growth inhibitors; and/or to dispensing of microflow of biological or chemical reagent at corresponding places where droplets containing active biological components are dispensed, dispensing of reagents occurring either during dispensing of the active biological components or by differed dispensing of other droplets on sites on which the droplets of active biological components have been deposited previously or will be deposited later.

39. Method for counting particles, using a device according to claim 1, fluid containing particles circulating in the first channel, the particles being counted by measuring means or optical means.

40. Device for dispensing droplets comprising:
a first channel, known as main channel, comprising two branches, with an input opening for circulation of a first fluid flow,
a second channel with an injection opening for circulation of a second fluid, comprising branches on either side of the main channel, and which terminates via an ejection orifice for part of the flow and another outlet orifice for another part of flow,
the branches of the first and second channel joining up in an intersection zone,
a series of electrodes for measuring a physical property of particles or cells in the first channel, matching electrodes being arranged on either side of a channel branch, and
means for generating a pressure wave in the second channel.

41. Device according to claim 40, wherein the electrodes are suitable for measuring optical properties of a fluid flow and/or the impedance of the medium and/or electric parameters of a fluid flow, and/or differential variation of impedance.

42. Device according to claim 40 further comprising an electrovalve and/or a physical-mechanical actuator for generating a pressure wave and/or a flow.
